(19) United States
(12) Patent Application Publication
(10) Pub. No.: US 2016/0015549 A1
(43) Pub. Date: Jan. 21, 2016

(54) PORTABLE CERVICAL TRACTION DEVICE

(71) Applicant: Hollywog, LLC, Chattanooga, TN (US)

(72) Inventors: Charles M. Thomas, Hixson, TN (US); D. Keith Hagy, Hixson, TN (US); Thu-Ha Thi Duncan, Cleveland, TN (US); John S. Marshall, Harrison, TN (US); Guy T. Hill, Rock Springs, GA (US); Herbert Allen Hughes, Ooltewah, TN (US); Christopher Shawn Hughes, Ooltewah, TN (US); Anita Hawkins, Cleveland, AL (US)

(73) Assignee: Hollywog, LLC, Chattanooga, TN (US)

(21) Appl. No.: 14/801,453

(22) Filed: Jul. 16, 2015

Related U.S. Application Data
(60) Provisional application No. 62/025,860, filed on Jul. 17, 2014.

(51) Int. Cl. A61F 5/055; A61F 5/37

(52) U.S. Cl. CPC A61F 5/055; A61F 5/3707

(57) ABSTRACT

A cervical traction device includes a base assembly having a sled frame and a sled assembly that is adapted to move along the sled frame. The device includes a plurality of unique head support cushions. The sled assembly includes a recess that is adapted to removably receive each of the plurality of head support cushions. The device also includes a shoulder support cushion and a halter assembly having a head halter that is adapted to be removably attached to the head of the patient. A linear actuator is attached between the base assembly and the sled assembly and adapted to move the sled assembly along the sled frame.

### CROSS-REFERENCE TO RELATED APPLICATION

This application claims the benefit of U.S. Provisional Patent Application No. 62/025,860, which was filed on Jul. 17, 2014.

### FIELD OF THE INVENTION

This invention relates generally to cervical traction devices. More specifically, the invention relates to a portable cervical traction device that may be placed on any suitable surface for use in applying traction forces to the cervical musculature and spine.

### BACKGROUND AND DESCRIPTION OF THE PRIOR ART

Traction devices are commonly used to relieve pressure on inflamed or pinched nerves. Cervical traction devices generally operate by applying a traction force to the head and/or neck of a patient, and lumbar traction devices generally operate by applying a traction force to the lumbar spine. Such devices can relieve pain in the neck and lower back by stretching the cervical and/or lumbar musculature and spine. Generally, traction devices operate by applying a series of traction or pulling forces to the head and/or neck or to the hips while the rest of the body is held down by straps or by friction forces on a table or other surface.

Portable traction devices are known for allowing patients to perform traction therapy on themselves in the privacy of their homes, or for allowing therapists to perform traction therapy in a clinical setting by placing the portable device on any of various inexpensive support tables or even on the floor. Common portable cervical traction devices apply traction forces by means of pneumatic cylinders that are controlled by a hand pump, spring-loaded linear actuators or motorized linear actuators. Such traction devices do not typically provide support for the patient's shoulders in such a way as to help to position the patient properly with respect to the traction apparatus. In addition, conventional cervical traction devices do not include a self-adjusting halter assembly for properly positioning the head and neck of a patient with respect to the patient's shoulders to obtain a maximum therapeutic benefit.

It would be desirable if a cervical traction device could be provided that includes multiple interchangeable self-storing head support cushions for use in positioning the patient's head and neck in any of several angular positions with respect to the patient's shoulders. It would also be desirable if the cervical traction device included a shoulder support assembly that assists in properly positioning the patient to receive traction therapy. It would also be desirable if the cervical traction device included a halter assembly that cooperates with the shoulder support assembly and one of the head support cushions for properly positioning the cervical spine of a patient to obtain a maximum therapeutic benefit.

### ADVANTAGES OF A PREFERRED EMBODIMENT OF THE INVENTION

Among the advantages of a preferred embodiment of the invention is that provides a cervical traction device having multiple interchangeable self-storing head support cushions for use in positioning the patient's head and neck in any of several angular positions with respect to the patient's shoulders. Another advantage of a preferred embodiment of the invention is that it includes a support component for the patient's shoulders that assists in properly positioning the patient to receive traction therapy. Still another advantage of a preferred embodiment of the invention is that it includes a halter assembly which cooperates with the shoulder support component and the selected head support cushion for properly positioning the cervical spine of a patient to obtain a maximum therapeutic benefit.

Additional objects and advantages of this invention will become apparent from an examination of the drawings and the ensuing description.

### Notes on Construction

The use of the terms “a”, “an”, “the” and similar terms in the context of describing the invention are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. The terms “substantially”, “generally” and other words of degree are relative modifiers intended to indicate permissible variation from the characteristic so modified. The use of such terms in describing a physical or functional characteristic of the invention is not intended to limit such characteristic to the absolute value which the term modifies, but rather to provide an approximation of the value of such physical or functional characteristic. All methods described herein can be performed in any suitable order unless otherwise specified herein or clearly indicated by context.

The use of any and all examples or exemplary language (e.g., “such as” and “preferably”) herein is intended merely to better illuminate the invention and the preferred embodiments thereof, and not to place a limitation on the scope of the invention. Nothing in the specification should be construed as indicating any element as essential to the practice of the invention unless so stated with specificity. Several terms are specifically defined herein. These terms are to be given their broadest possible construction consistent with such definitions, as follows:

The terms “above”, “upper” and similar terms, when used with respect to a traction device or a component of a traction device, refer to a relative location or direction away from the surface on which the device is placed during operation.

The terms “below”, “lower” and similar terms, when used with respect to a traction device or a component of a traction device, refer to a relative location or direction towards the surface on which the device is placed during operation.

The term “rear” and similar terms, when used with respect to a traction device or a component of a traction device, refer to a relative location or direction towards the end of the device on which the shoulder support cushion is located.

The terms “front”, “forward” and similar terms, when used with respect to a traction device or a component of a traction device, refer to a relative location or direction away from the end of the device on which the shoulder support cushion is located.

The term “linear actuator” refers to an electric, hydraulic, electro-hydraulic or mechanical device that generates force which is directed in a straight line.

### SUMMARY OF THE INVENTION

The invention comprises a cervical traction device for use in applying cervical traction to a patient. This cervical traction device includes a base assembly and a sled assembly. The base assembly has a sled frame, and the sled assembly is adapted to move along the sled frame. A linear actuator is attached between the base assembly and the sled assembly, and adapted to move the sled assembly along the sled frame. The sled assembly also has a recess that is adapted to removably receive each of a plurality of head support cushions. Each of the head support cushions has a configuration that is different from the configurations of the other head support cushions. The cervical traction device also includes a shoulder support cushion, and a halter assembly that is adapted to be removably attached to the head of the patient. The shoulder support cushion, head support cushions and halter assembly cooperate to position the head and neck of the patient with respect to the patient's shoulders to facilitate the application of a therapeutic traction force.

In a preferred embodiment of the invention, the shoulder support cushion is removably attached to the base assembly and includes a storage slot that is adapted to securely hold one or more of the head support cushions. The halter assembly includes left and right halter supports which are attached on opposite sides of the sled assembly, and a head halter that is adapted to be removably attached to the halter supports and to the head of the patient. The preferred embodiment of the invention allows for the application of a traction force to a patient by movement of the sled assembly with respect to the base assembly that will provide a release of the cervical spine. The preferred halter assembly is sized and configured so as to be self-adjusting to accommodate patients of various sizes and weights.

In order to facilitate an understanding of the invention, the preferred embodiments of the invention is illustrated in the drawings, and a detailed description thereof follows. It is not intended, however, that the invention be limited to the particular embodiments described or to use in connection with the apparatus illustrated herein. Various modifications and alternative embodiments such as would ordinarily occur to one skilled in the art to which the invention relates are also contemplated and included within the scope of the invention described and claimed herein.

### BRIEF DESCRIPTION OF THE DRAWINGS

The presently preferred embodiments of the invention are illustrated in the accompanying drawings, in which like reference numerals represent like parts throughout, and in which:

FIG. 1 is a perspective view of a preferred embodiment of the cervical traction device of the invention, with the head halter removed to more clearly reveal the features of the halter supports.

FIG. 2 is a top view of the embodiment of the cervical traction device shown in FIG. 1.

FIG. 3 is a side view of the embodiment of the cervical traction device shown in FIGS. 1 and 2.

FIG. 4 is an end view of the embodiment of the cervical traction device shown in FIGS. 1-3, taken along line 4-4 of FIG. 3.

FIG. 5 is an end view of the embodiment of the cervical traction device shown in FIGS. 1-4, taken along line 5-5 of FIG. 3.

FIG. 6 is an exploded view, in perspective, of the cervical traction device of the invention shown in FIGS. 1-5.

FIG. 7 is an exploded view, in perspective, of a portion of the base assembly of the cervical traction device shown in FIGS. 1-6.

FIG. 8 is an exploded view, in perspective, of the sled assembly of the cervical traction device shown in FIGS. 1-6.

FIG. 9A is a top view of a first head support cushion that is included within a preferred embodiment of the invention.

FIG. 9B is a side view of the head support cushion shown in FIG. 9A.

FIG. 9C is an end view of the head support cushion shown in FIGS. 9A and 9B.

FIG. 9D is a perspective view of the head support cushion shown in FIGS. 9A-9C.

FIG. 9E is a bottom view of the head support cushion shown in FIGS. 9A-9D.

FIG. 9F is a sectional view of the head support cushion shown in FIGS. 9A-9E, taken along line F-F of FIG. 9E.

FIG. 10A is a top view of a second head support cushion that is included within a preferred embodiment of the invention.

FIG. 10B is a side view of the head support cushion shown in FIG. 10A.

FIG. 10C is an end view of the head support cushion shown in FIGS. 10A and 10B.

FIG. 10D is a perspective view of the head support cushion shown in FIGS. 10A-10C.

FIG. 10E is a bottom view of the head support cushion shown in FIGS. 10A-10D.

FIG. 10F is a sectional view of the head support cushion shown in FIGS. 10A-10E, taken along line F-F of FIG. 10E.

FIG. 11A is a top view of a third head support cushion that is included within a preferred embodiment of the invention.

FIG. 11B is a side view of the head support cushion shown in FIG. 11A.

FIG. 11C is an end view of the head support cushion shown in FIGS. 11A and 11B.

FIG. 11D is a perspective view of the head support cushion shown in FIGS. 11A-11C.

FIG. 11E is a bottom view of the head support cushion shown in FIGS. 11A-11D.

FIG. 11F is a sectional view of the head support cushion shown in FIGS. 11A-11E, taken along line F-F of FIG. 11E.

FIG. 12A is a top view of the preferred shoulder support cushion that is a part of the invention.

FIG. 12B is a side view of the shoulder support cushion shown in FIG. 12A.

FIG. 12C is a front end view of the shoulder support cushion shown in FIGS. 12A and 12B.

FIG. 12D is a perspective view of the shoulder support cushion shown in FIGS. 12A-12C.

FIG. 12E is a bottom view of the shoulder support cushion shown in FIGS. 12A-12D.

FIG. 12F is a sectional view of the shoulder support cushion shown in FIGS. 12A-12E, taken along line F-F of FIG. 12A.

FIG. 12G is a sectional view of the shoulder support cushion shown in FIGS. 12A-12F, taken along line G-G of FIG. 12E.

FIG. 12H is a sectional view of the shoulder support cushion shown in FIGS. 12A-12G, taken along line H-H of FIG. 12E.

FIG. 13A is an illustration of a first embodiment of a head halter that is a part of the preferred embodiment of the invention.

FIG. 13B is an illustration of the head halter shown in FIG. 13A, showing how it is attached to a patient and to the cervical traction device shown in FIGS. 1-6.

FIG. 14A is an illustration of a second embodiment of a head halter that is a part of the preferred embodiment of the invention.

FIG. 14B is an illustration of the head halter shown in FIG. 14A, showing how it is attached to a patient and to the cervical traction device shown in FIGS. 1-6.

### DESCRIPTION OF THE PREFERRED EMBODIMENT OF THE INVENTION

A preferred embodiment 20 of a cervical traction device is illustrated in the drawings. As shown therein, cervical traction device 20 includes base assembly 22, sled assembly 24, left and right halter supports 26 and 28 respectively, and shoulder support cushion 30.

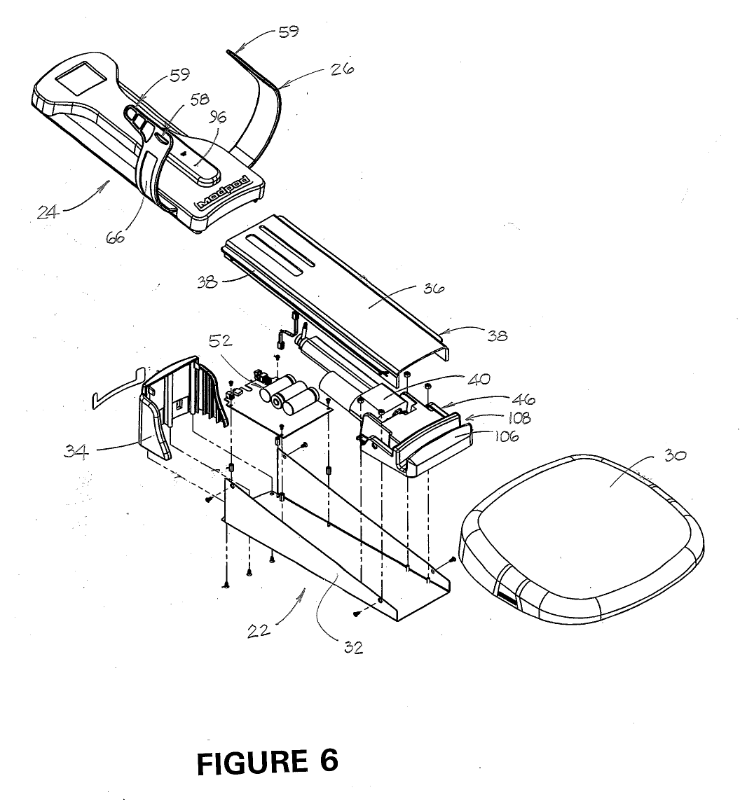

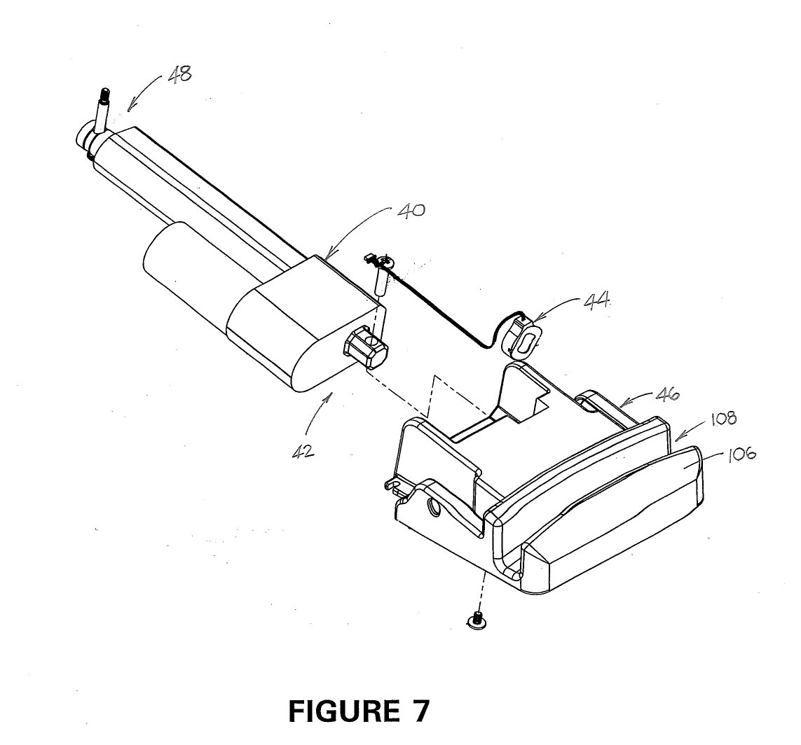

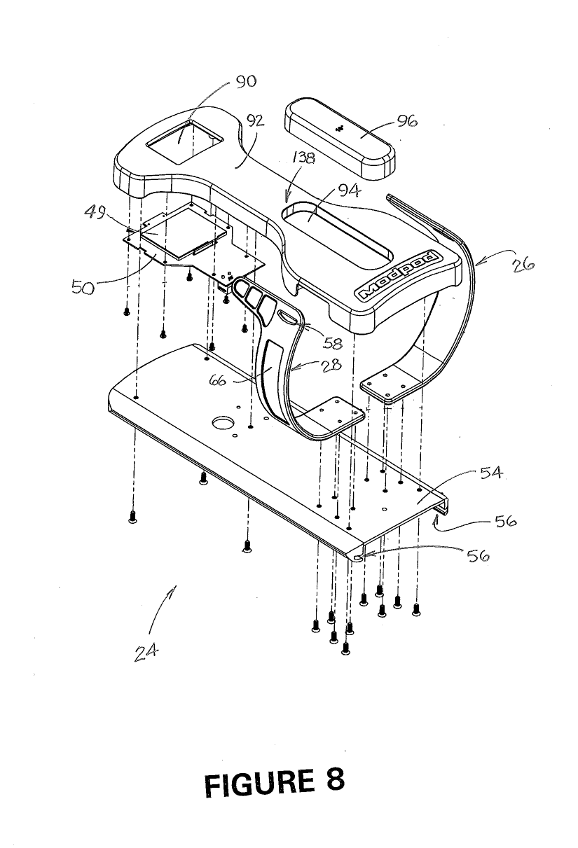

As shown in FIG. 6, base assembly 22 includes base frame 32, front closure 34, and sled frame 36. Two low-friction sled runners 38 are attached to the sled frame to facilitate movement of sled assembly 24 on the sled frame. Preferably, sled runners 38 are comprised of a blend of polycarbonate and glass fibers. Enclosed within base assembly 22 is linear actuator 40 (best shown in FIG. 7). Rear end 42 of linear actuator 40 is attached to load cell assembly 44 and to rear support 46 of the base assembly. Front end 48 of linear actuator 40 is attached to sled assembly 24. The load cell assembly is operatively attached to the linear actuator and to controller and interactive display assembly 49 located on controller base 50 (shown in FIG. 8) in sled assembly 24. Actuator 40 is powered by rechargeable battery pack 52 located in base frame 32.

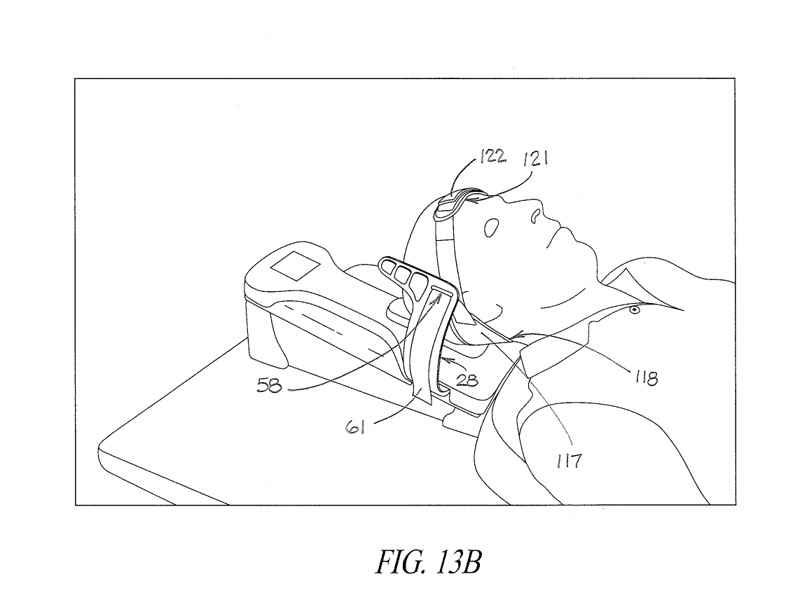

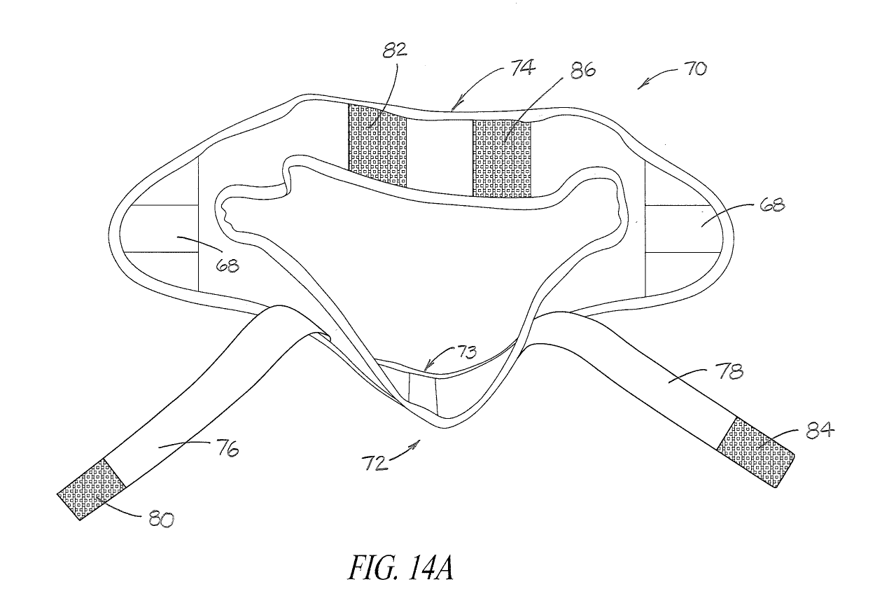

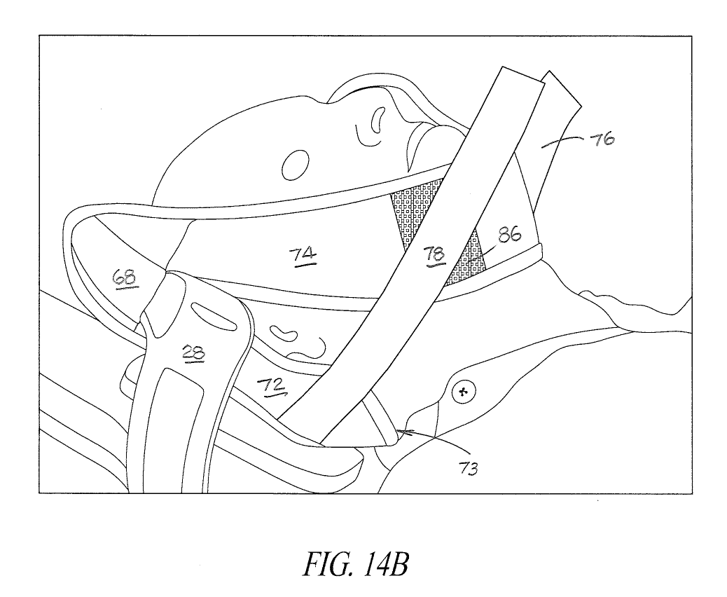

As shown in FIG. 8, sled assembly 24 includes sled base 54, which is provided with a pair of slots 56 for engaging sled runners 38 on sled frame 36. Attached to the sled base is left halter support 26 and right halter support 28. Each of the halter supports has a slot 58 and a front projection 59. Each of slots 58 is adapted to receive an end strap 60 or 61 of head halter 62 (shown in FIGS. 13A and 13B). Each end strap is preferably provided with a portion 64 of a hook and loop fastener, and this portion of the hook and loop fastener engages a mating portion 66 of a hook and loop fastener located on the halter support to hold head halter 62 in place with respect to the halter supports. Each of front projections 59 is adapted to receive cupped end 68 of head halter 70 (shown in FIGS. 14A and 14B).

Controller and interactive display assembly 49 allows a user of cervical traction device 20 to select from and modify any of various programmed clinical protocols or to program additional clinical protocols for the application of traction forces, and to operate device 20 to apply the selected traction forces to a patient. The interactive display portion of assembly 49 is visible through window 90 of sled top 92 when the sled top is attached to sled base 54. In this embodiment of the invention, the controller and interactive display assembly continuously measures the distance traveled by the sled and cooperates with load cell assembly 44 to measure the load or traction force applied by the linear actuator to determine the proper application of traction forces for maximum therapeutic effect, as set by means of the interactive display portion of assembly 49. This allows cervical traction device 20 to continuously apply the desired amount of traction force, even if the patient moves his or her head during treatment.

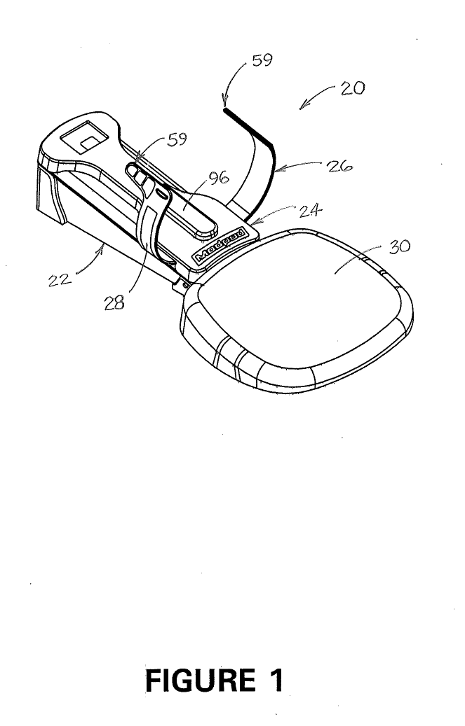

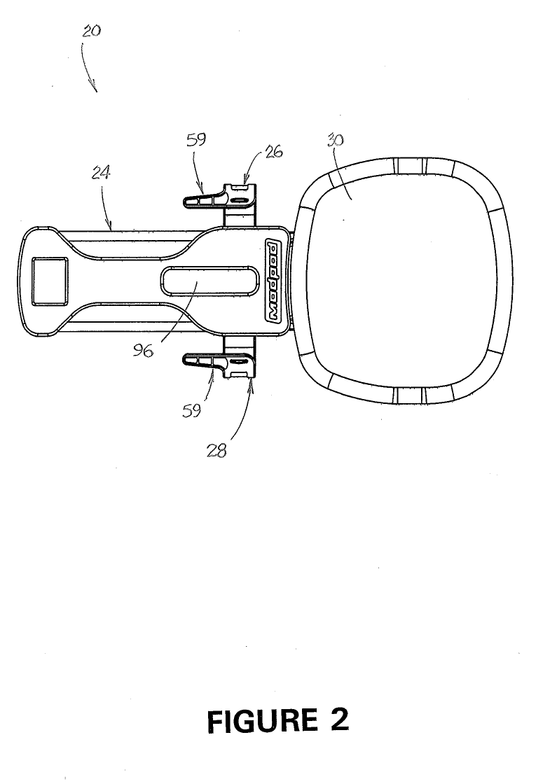

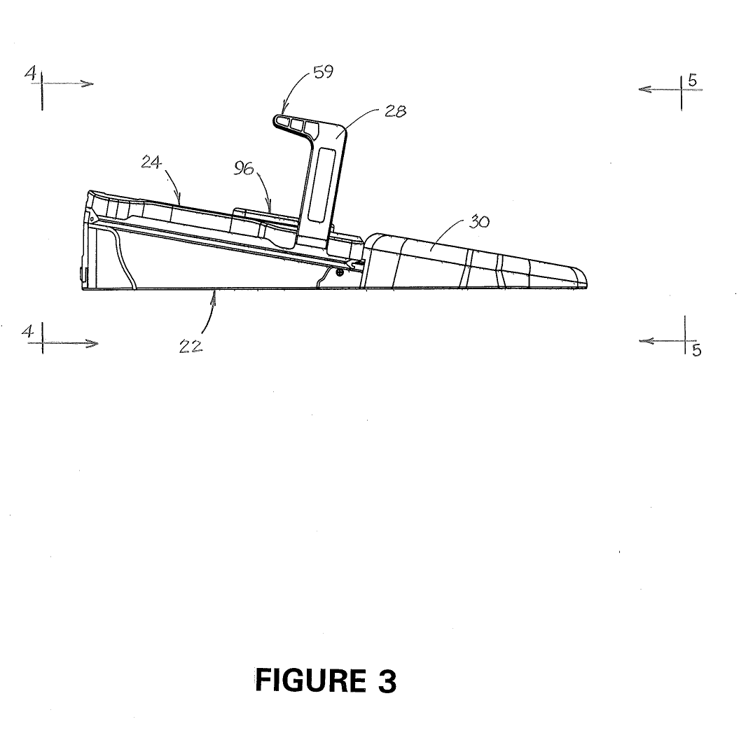

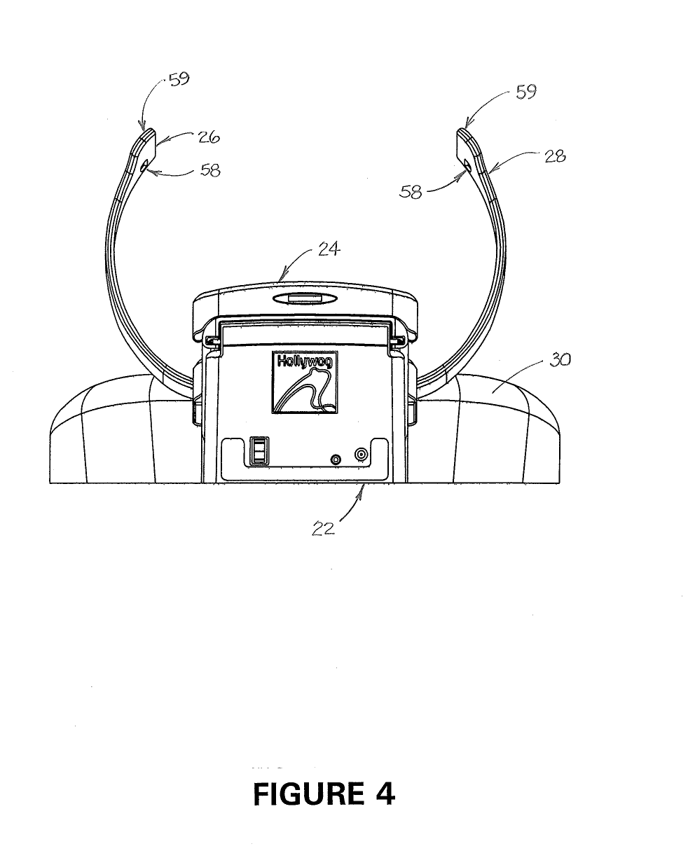

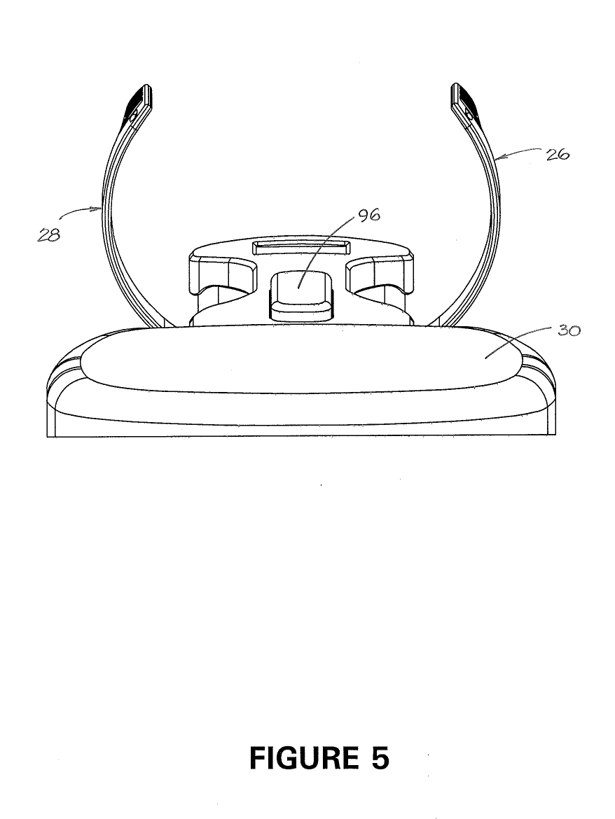

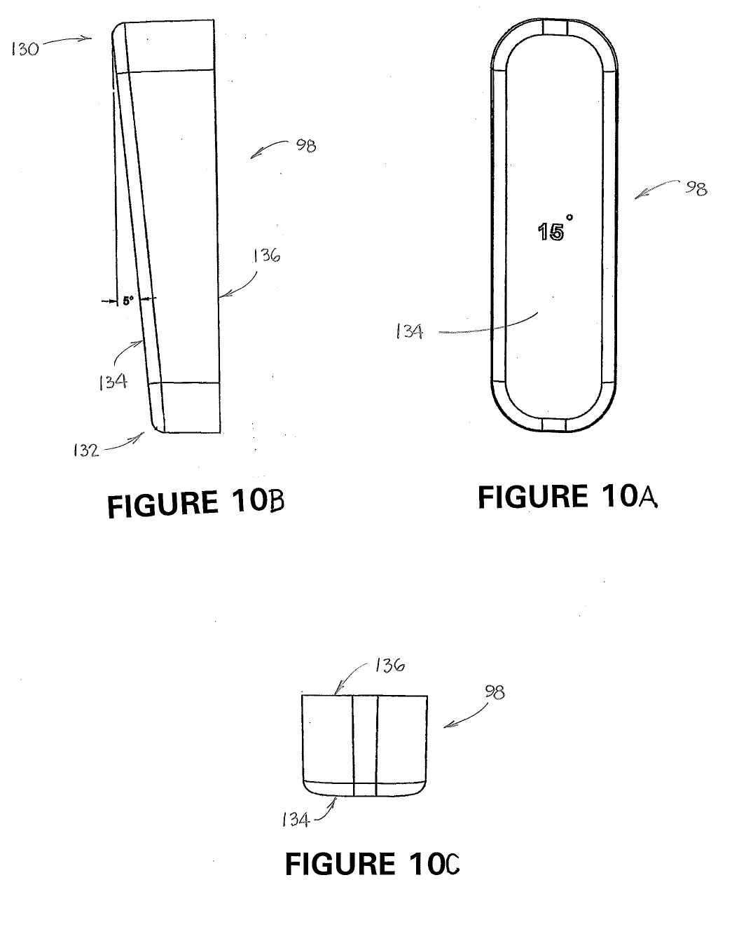

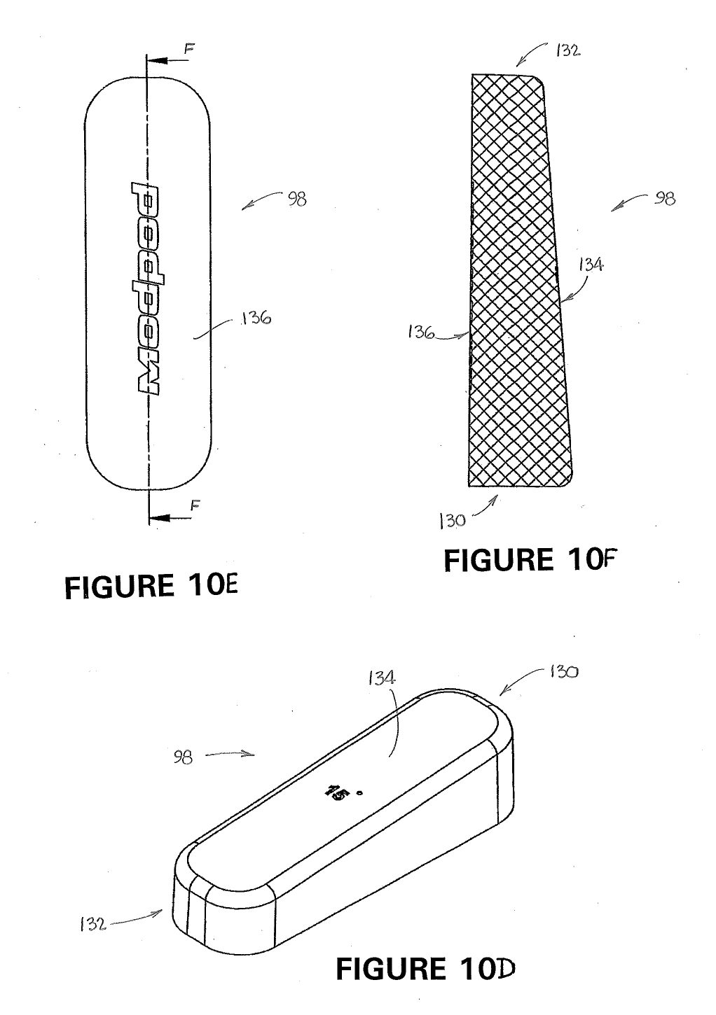

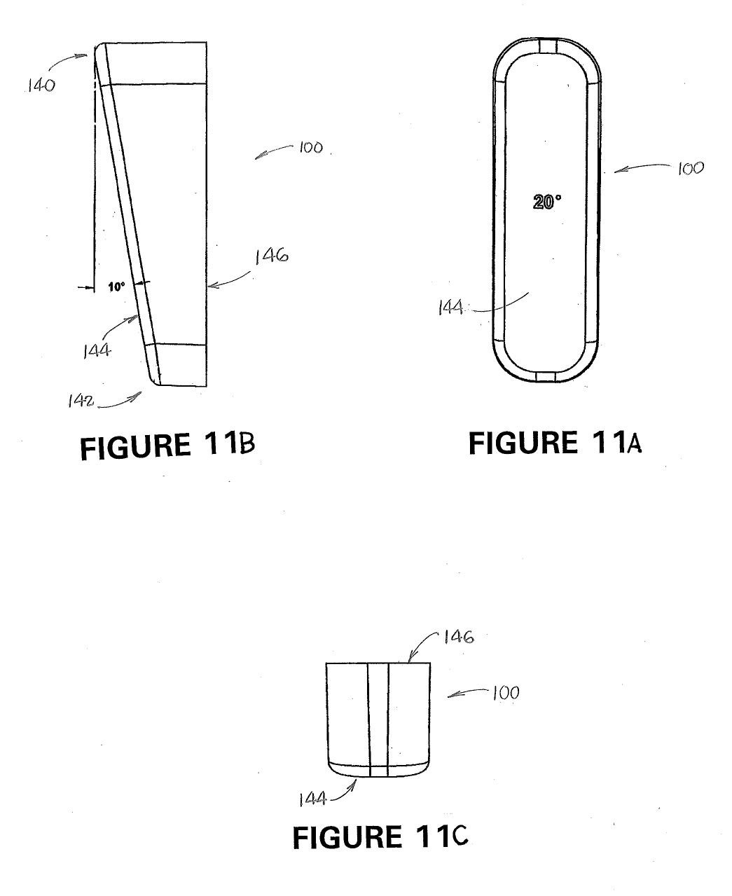

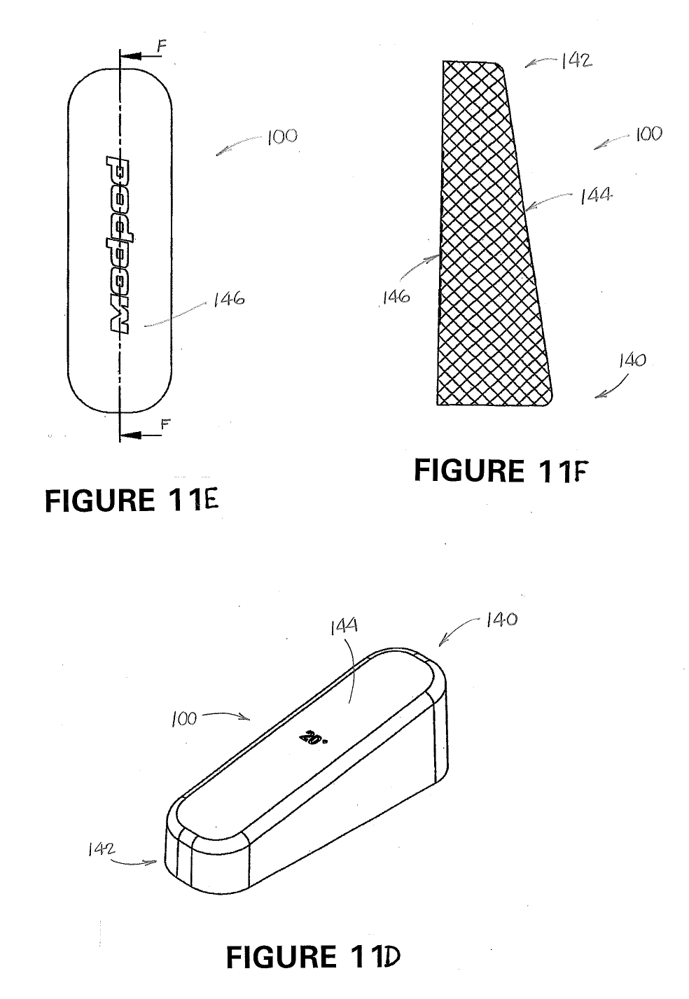

Also provided in sled top 92 is cut-out 94 which forms, in cooperation with sled base 54, a recess in the sled assembly that is adapted to removably receive any of several head support cushions, such as head support cushion 96 shown in FIGS. 1-3, 5, 6, 8 and 9A-9E, head support cushion 98 shown in FIGS. 10A-10F and head support cushion 100 shown in FIGS. 11A-11F.

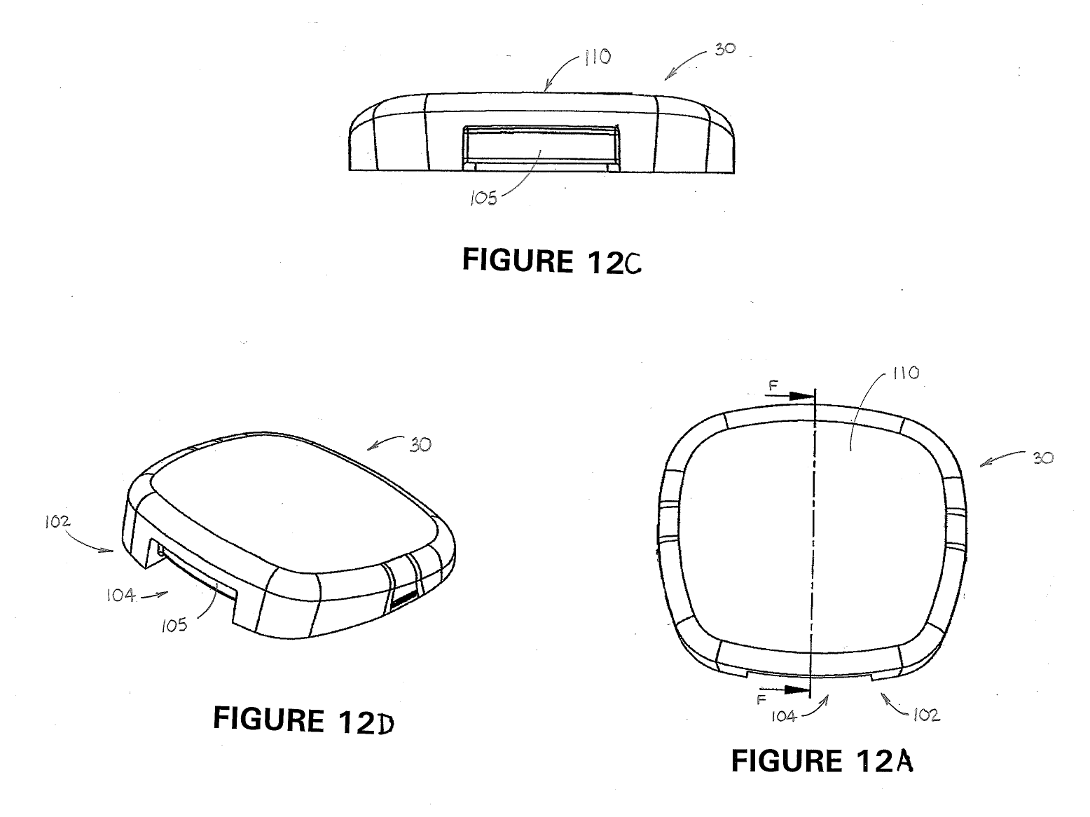

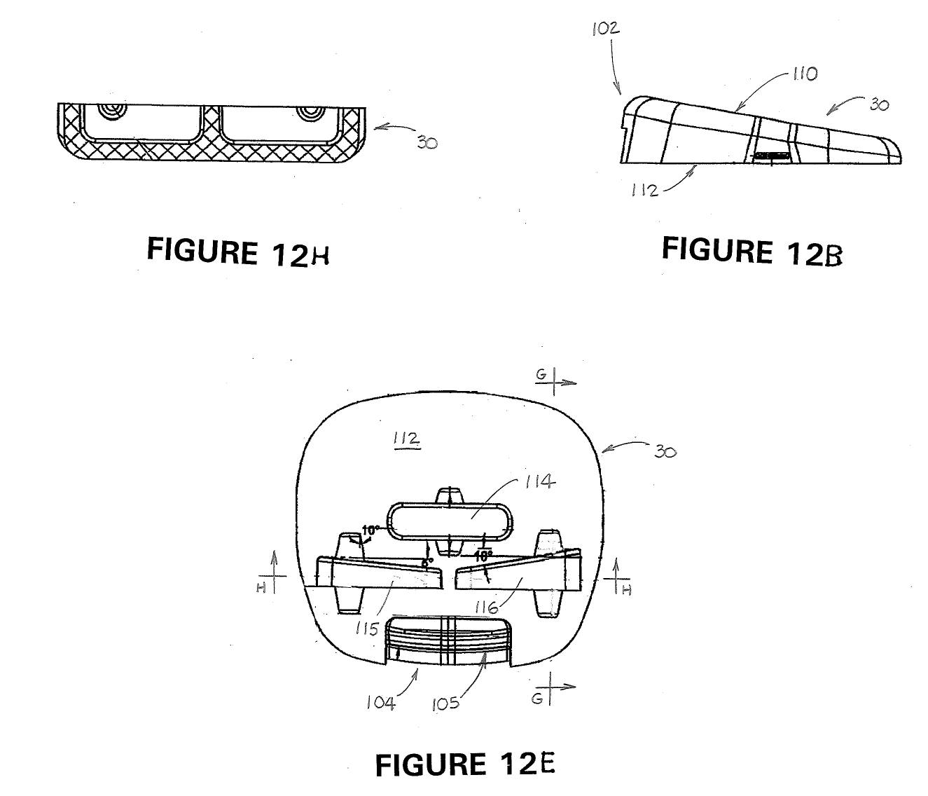

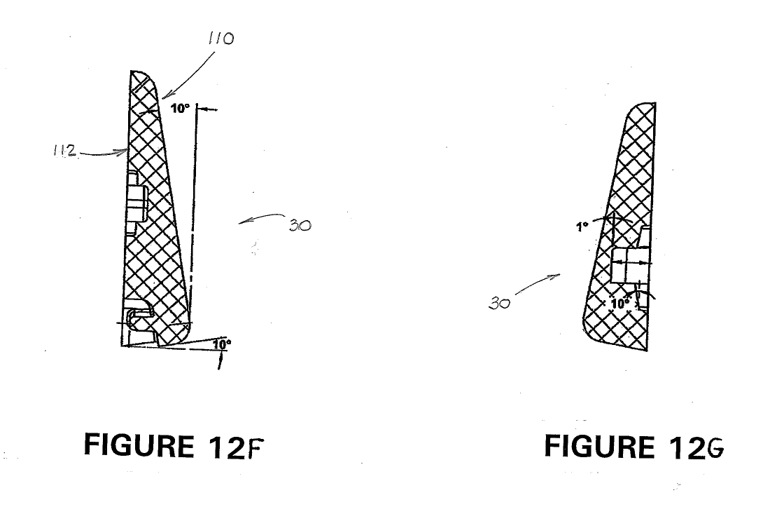

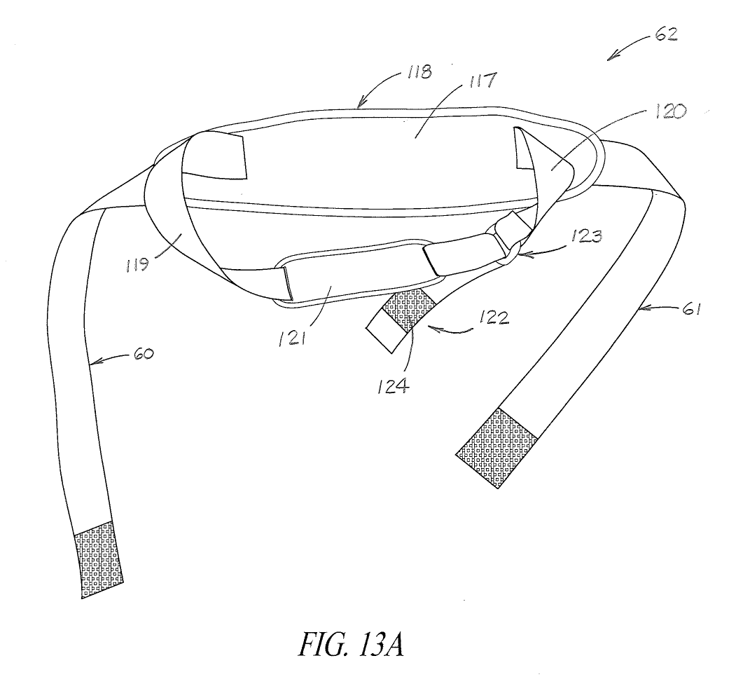

The invention also includes removable shoulder support cushion 30 having a front end 102 with an opening 104 and a base engagement projection 105 recessed in the opening, as shown in FIGS. 12A-12E. FIGS. 6 and 7 illustrate nose portion 106 and slot 108 of rear support 46 of base assembly 22. By comparing FIGS. 6, 7 and 12A-12E, it can be seen that base engagement projection 105 is sized and configured to be received in slot 108 in front of nose portion 106 of rear support 46 to securely hold the shoulder support cushion in engagement with the base assembly with the shoulder support cushion resting on the surface on which device 20 is placed. The upper surface 110 of shoulder support cushion is inclined upwardly towards front end 102, preferably by 10° with respect to lower surface 112, as best shown in FIG. 12F. Lower surface 112 also includes storage slot 114 that is adapted to securely hold head support cushion 96, storage slot 115 that is adapted to securely hold head support cushion 98, and storage slot 116 that is adapted to securely hold head support cushion 100, when the head support cushions are not placed in the recess defined in party by cut-out 94.

The invention includes a self-adjusting halter assembly for properly positioning the head and neck of a patient with respect to the patient's shoulders to obtain a maximum therapeutic benefit. The halter assembly is also sized and configured so as to be self-adjusting to accommodate patients of various sizes and weights. The halter assembly comprises left halter support 26, right halter support 28 and a head halter. Two different head halters are described herein. Head halter 62 is particularly adapted for use by patients with temporomandibular joint (“TMJ”) disorders or other disorders that would cause discomfort if a head halter included a chin strap. Head halter 70 is adapted for use by patient's who can easily tolerate a chin strap.

Head halter 62 includes left end strap 60 and right end strap 61, neck support strap 117 having rear edge 118, and an adjustable forehead support strap comprising left side portion 119 and right side portion 120, both of which are attached to the neck support strap. Left side portion 119 also includes forehead pad 121 and free end 122. Right side portion 120 terminates in ring 123. Free end 122 of left side portion 119 is provided with a portion 124 of a hook and loop fastener that is adapted to engage and mate with a portion of a hook and loop fastener (not shown) located on the left side portion 119 adjacent forehead pad 121 after free end 122 is passed through ring 123. The proper placement of head halter 62 on the left and right halter supports is shown in FIG. 13B, with the end straps (only one of which, end strap 61, is shown in FIG. 13B) being passed through slots 58 in the halter supports and secured to the halter supports as described above. Left halter support 26, right halter support 28 and head halter 62 are sized and configured so that when end straps 60 and 61 of head halter 62 are placed through slots 58 in the halter supports (as shown in FIG. 13B) and secured to the halter supports, and the patient is positioned so that rear edge 118 of neck support strap 117 is at or slightly beneath the occiput and the free end 122 of left side portion 119 of adjustable forehead support strap is passed through ring 123 and secured around the patient's forehead, the application of a traction force to the patient by movement of sled assembly 24 on the sled frame will provide a C1-C7 release of the cervical spine.

Head halter 70 also includes back strap 72 having rear edge 73, chin strap 74, and a pair of fastener straps 76 and 78. Fastener strap 76 is provided with a portion 80 of a hook and loop fastener, and this portion of the hook and loop fastener engages a mating portion 82 of a hook and loop fastener located on chin strap 74. Similarly, fastener strap 78 is provided with a portion 84 of a hook and loop fastener, and this portion of the hook and loop fastener engages a mating portion 86 of a hook and loop fastener located on chin strap 74. After the cupped ends 68 of head halter 70 are slipped over the front projections on the halter supports, the patient is positioned so that rear edge 73 of back strap 72 is at or slightly beneath the occiput and chin strap 74 is placed under the patient's jaw. Then, the fastener straps are attached to the chin strap to snugly hold the patient's head in a suitable position for the application of traction therapy.

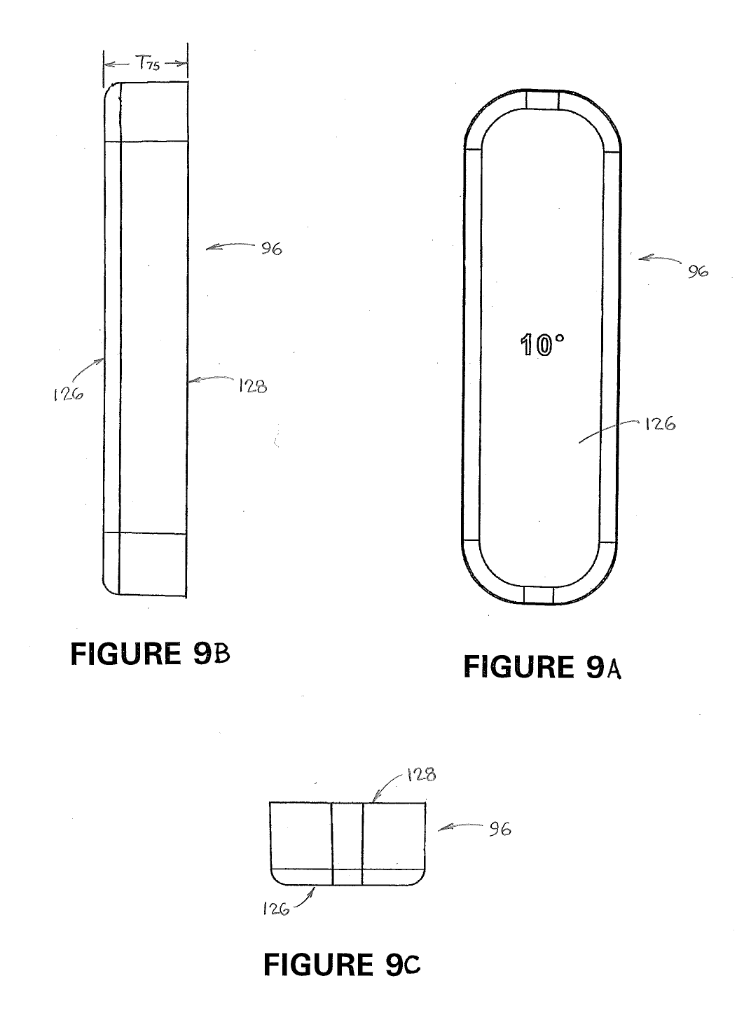

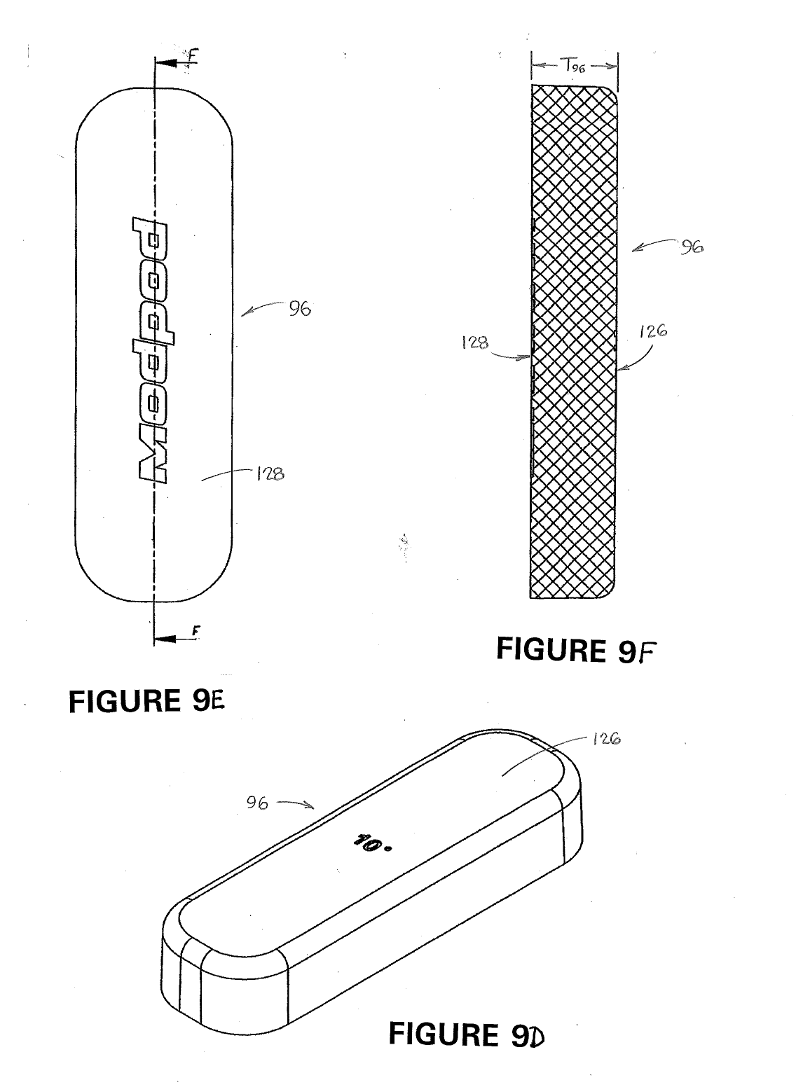

As shown in FIGS. 9A-9F, head support cushion 96 has upper surface 126 that is generally parallel to lower surface 128, so that the thickness T₉₆ is generally constant along the length of the head support cushion. When head support cushion 96 is properly inserted into the recess defined in part by cut-out 94 (as shown in FIG. 1, for example), and a patient is positioned with his shoulders on shoulder support cushion 30, his head on head support cushion 96 between left halter support 26 and right halter support 28, and his neck supported by head halter 62 or head halter 70 as described above, the angle of the traction force applied by movement of sled frame 36 on sled base 54 with respect to the surface on which most of the patient's body rests will be approximately 10°.

As shown in FIGS. 10A-10F, head support cushion 98 has a front end 130 and a rear end 132. The upper surface 134 of head support cushion 98 is inclined upwardly towards front end 130, preferably by 5° with respect to lower surface 136, as best shown in FIG. 10B. Consequently, when head support cushion 98 is properly inserted into the recess defined in party by cut-out 94, with front end 130 towards front end 138 of cut-out 94 (shown in FIG. 8), and a patient is positioned with his shoulders on shoulder support cushion 30, his head on head support cushion 98 between left halter support 26 and right halter support 28, and his neck supported by head halter 62 or head halter 70 as described above, the angle of the traction force applied by movement of sled frame 36 on sled base 54 with respect to the surface on which most of the patient's body rests will be approximately 10°+5°=15°.

As shown in FIGS. 11A-11F, head support cushion 100 has a front end 140 and a rear end 142. The upper surface 144 of head support cushion 100 is inclined upwardly towards front end 140 preferably by 10° with respect to lower surface 146, as best shown in FIG. 11B. Consequently, when head support cushion 100 is properly inserted into the recess defined in part by cut-out 94, with front end 140 towards front end 138 of cut-out 94, and a patient is positioned with his shoulders on shoulder support cushion 30, his head on head support cushion 100 between left halter support 26 and right halter support 28, and his neck supported by head halter 62 or head halter 70 as described above, the angle of the traction force applied by movement of sled frame 36 on sled base 54 with respect to the surface on which most of the patient's body rests will be approximately 10°+10°=20°.

Head support cushions that cooperate with the halter assembly and the shoulder support cushion to provide different angles of application of a traction force from those specifically described herein are contemplated within the scope of the invention.

Although this description contains many specifics, these should not be construed as limiting the scope of the invention but as merely providing illustrations of some of the presently preferred embodiments thereof, as well as the best mode contemplated by the inventors of carrying out the invention. The invention, as described herein, is susceptible to various modifications and adaptations as would be appreciated by those having ordinary skill in the art to which the invention relates.

What is claimed is:

1. A cervical traction device for use in applying cervical traction to a patient, said cervical traction device comprising:
(a) a base assembly having a sled frame;
(b) a plurality of head support cushions, each of which has a configuration that is different from the configurations of the other head support cushions;
(c) a sled assembly:
(i) that is adapted to move along the sled frame;
(ii) including a recess that is adapted to removably receive each of the plurality of head support cushions;
(d) a shoulder support cushion;
(e) a linear actuator that is:
(i) attached between the base assembly and the sled assembly;
(ii) adapted to move the sled assembly along the sled frame;
(f) a halter assembly including a head halter that is adapted to be removably attached to the head of the patient.

2. The cervical traction device of claim 1 wherein the plurality of head support cushions, the shoulder support cushion and the halter assembly are configured and arranged so that each of the head support cushions is employed in cooperation with the shoulder support cushion and the halter assembly to position the head and neck of the patient with respect to the patient's shoulders at a particular angle.

3. The cervical traction device of claim 1 which includes:
(a) a first head support cushion having:
(i) a front end;
(ii) a rear end;
(iii) a lower surface;
(iv) an upper surface that is generally parallel to the lower surface;
(b) a second head support cushion having:
(i) a front end;
(ii) a rear end;
(iii) a lower surface;
(iv) an upper surface that is inclined upwardly towards the front end by about 5° with respect to the lower surface;
(c) a third head support cushion having:
(i) a front end;
(ii) a rear end;
(iii) a lower surface;
(iv) an upper surface that is inclined upwardly towards the front end by about 10° with respect to the lower surface.

4. The cervical traction device of claim 1 wherein:
(a) the base assembly comprises a sled frame having a pair of low-friction sled runners;
(b) the sled assembly includes a sled base having a pair of slots that are adapted to engage the sled runners on the sled frame.

5. The cervical traction device of claim 1 wherein:
(a) the halter assembly comprises:
(i) a left halter support;
(ii) a right halter support;
(b) the head halter is adapted to be removably attached to the left and right halter supports.

6. The cervical traction device of claim 5 wherein:
(a) the head halter comprises:
(i) a pair of end straps;
(ii) a neck support strap having a rear edge;
(iii) a forehead support strap;
(b) each of the halter supports has a slot that is adapted to receive an end strap of the head halter.

7. The cervical traction device of claim 6 wherein the forehead support strap is adjustable and comprises:
(a) a left side portion that is attached to the neck support pad and includes:
(i) a forehead pad;
(ii) a free end that is provided with a portion of a hook and loop fastener that is adapted to engage and mate with a portion of a hook and loop fastener on the left side portion adjacent the forehead pad;
(b) a right side portion that is attached to the neck support strap and terminates in a ring.

8. The cervical traction device of claim 5 wherein:
(a) the head halter comprises:
(i) a back strap;
(ii) a chin strap;
(iii) a pair of fastener straps;
(iv) a pair of cupped ends;
(b) each of the halter supports has a front projection that is adapted to receive a cupped end of the head halter.

9. The cervical traction device of claim 1 wherein the shoulder support cushion is removably attached to the base assembly.

10. The cervical traction device of claim 9 wherein:
(a) the base assembly includes:
(i) a rear support having a nose portion;
(ii) a slot that is located in front of the nose portion;
(b) the shoulder support cushion has a front end with an opening and a base engagement projection recessed in the opening, said base engagement projection being sized and configured to be received in the slot of the base assembly behind the nose portion.

11. The cervical traction device of claim 1 wherein the shoulder support cushion comprises:
(a) a front end;
(b) a rear end;
(c) a lower surface that includes a plurality of storage slots, each of which is adapted to hold a head support cushion;
(d) an upper surface that is inclined upwardly towards the front end with respect to the lower surface.

12. The cervical traction device of claim 11 wherein the upper surface of the shoulder support cushion is inclined upwardly towards the front end by about 10° with respect to the lower surface.

13. The cervical traction device of claim 1, which includes:
(a) a controller and interactive display assembly that is operatively attached to the linear actuator;
(b) a load cell assembly that is operatively attached to the linear actuator and to the controller and interactive display assembly.

14. The cervical traction device of claim 13 wherein the controller and interactive display assembly:
(a) is adapted to continuously measure the distance traveled by the sled assembly along the sled frame;
(b) cooperates with the load cell assembly to measure the traction force applied by the linear actuator.

15. A method for applying traction to a patient, said method comprising:
(a) providing a cervical traction device comprising:
(i) a base assembly having a sled frame;
(ii) a plurality of head support cushions, each of which has a configuration that is different from the configurations of the other head support cushions;
(iii) a sled assembly that is adapted to move along the sled frame, said sled assembly including a recess that is adapted to removably receive each of the plurality of head support cushions;
(iv) a shoulder support cushion;
(v) a linear actuator that has a front end that is attached to the sled assembly and a rear end that is attached to the base assembly, said linear actuator being adapted to move the sled assembly along the sled frame;
(vi) a load cell assembly that is attached to the linear actuator and adapted to measure the traction force generated by the linear actuator;
(vii) a controller and interactive display assembly that is operatively attached to the load cell assembly and the linear actuator and adapted to control the movement of the sled assembly along the sled frame;
(viii) a halter assembly including a head halter that is adapted to be removably attached to the head of the patient;
(b) selecting one of the head support cushions;
(c) placing the selected head support cushion into the recess in the sled assembly;
(d) placing the patient so that the patient's shoulders are supported by the shoulder support cushion and the patient's head is supported by the selected head support cushion;
(e) attaching the head halter to the patient's head;
(f) operating the linear actuator to move the sled assembly along the sled frame.

16. The method of claim 15 which includes providing a cervical traction device:
(a) in which the controller and interactive display assembly:
(i) is adapted to continuously measure the distance traveled by the sled assembly along the sled frame;
(ii) cooperates with the load cell assembly to measure the traction force applied by the linear actuator;
(b) wherein the controller and interactive display assembly allows a user of the cervical traction device to select from, add to or modify any of various clinical protocols for the application of traction forces, and to operate the cervical traction device to apply the selected traction forces to a patient.

17. The method of claim 15 which includes providing a cervical traction device wherein:
(a) the halter assembly comprises:
(i) a left halter support;
(ii) a right halter support;
(b) the head halter is adapted to be removably attached to the left and right halter supports.

18. The method of claim 17 which includes:
(a) providing a cervical traction device wherein:
(i) the head halter comprises a left end strap, a right end strap, a neck support strap having a rear edge and a forehead support strap;
(ii) each of the halter supports has a slot that is adapted to receive an end strap of the head halter;
(b) wherein the left end strap of the head halter is passed through the slot in the left halter support;
(c) wherein the right end strap of the head halter is passed through the slot in the right halter support;
(d) wherein the patient is positioned so that:
(i) the patient's shoulders are supported by the shoulder support cushion, the patient's head is supported on the head support cushion between the left halter support and the right halter support;
(ii) the rear edge of the neck support strap is at or slightly beneath the occiput and the forehead support strap is secured around the patient's forehead.

19. The method of claim 17 which includes:
(a) providing a cervical traction device wherein:
(i) the head halter comprises a back strap having a rear edge, a chin strap that is provided with left and right portions of a hook and loop fastener, a left fastener strap that is provided with a portion of a hook and loop fastener that is adapted to engage and mate with left portion of the hook and loop fastener on the chin strap, a right fastener strap that is provided with a portion of a hook and loop fastener that is adapted to engage and mate with right portion of the hook and loop fastener on the chin strap, a left cupped end and a right cupped end;
(ii) each of the halter supports has a front projection that is adapted to receive a cupped end of the head halter;
(b) wherein the left cupped end of the head halter is placed over the front projection on the left halter support;
(c) wherein the right cupped end of the head halter is placed over the front projection on the right halter support;
(d) wherein the patient is positioned so that:
(i) the patient's shoulders are supported by the shoulder support cushion, the patient's head is supported on the head support cushion between the left halter support and the right halter support;
(ii) the rear edge of the back strap is at or slightly beneath the occiput, the chin strap of the head halter is placed under the patient's jaw, and the left fastener strap is attached to the chin strap and the right fastener strap is attached to the chin strap.
